(19) United States
(12) Patent Application Publication
(10) Pub. No.: US 2005/0249650 A1
(43) Pub. Date: Nov. 10, 2005

(54) Immersion retort

(75) Inventors: Eduard Hendrikus Johannes Damhuis, Bousval (BE); Richard David Eleew, Madisonville, LA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: FMC Technologies, Inc.

(21) Appl. No.: 10/842,023

(22) Filed: May 7, 2004

(51) Int. Cl. A61L 2/07; A23L 3/10

(52) U.S. Cl. 422/295; 422/302; 99/483; 426/521

(57) ABSTRACT

An overpressure retort system includes a process vessel in fluid flow communication with a storage vessel. Heated process fluid from the storage vessel is transferred to the process vessel, including through spraying outlets or nozzles to direct heated process fluid onto the nutrient product containers. After the cooking process has been completed, the heated process fluid is returned to the storage vessel and then cool process fluid is used to cool the nutrient products in the process vessel, including by spraying the cooled process fluid onto the nutrient product containers. During the cooking and cooling processes, the process fluid from the process vessel is recirculated, and the overpressure within the process vessel is controlled by supplying compressed gas at a controlled pressure to the headspace of the process vessel. The headspace of the process vessel and the headspace of the storage vessel are in fluid flow communication. In the cooling process, process fluid may be withdrawn from the process vessel, then cooled in a heat exchanger, then reintroduced to the process vessel.

### FIELD OF THE INVENTION

The present invention relates to retort systems for in-container preservation of nutritional products, more particularly to full or partial immersion retorts employing overpressure process.

### BACKGROUND OF THE INVENTION

In-container sterilization of nutritional products has been carried out either in batch or continuous pressure sterilizers. The batch systems consist of using one or more retorts in which a load of containers is treated. The treatment generally follows a time, pressure, and temperature profile that is pre-defined so that the containers located in the “coldest region” of the load will still be subject to a sufficient lethality in order to ensure that the nutritional product inside is rendered wholesome, while preserving the consumption characteristics of the product, such as texture and taste.

Various types of retorts and retort processes are utilized, including full or partial immersion of the containers in a fluid process medium, typically superheated water. A full immersion retort system normally consists of a process vessel and a storage vessel. The nutritional product containers are held in baskets or cages that are loaded into the process vessel and then rotated within the process vessel. Such rotation can be end-over-end by a reel or drum apparatus inside the retort process vessel driven by a variable speed drive motor. The process fluid within the storage vessel is preheated, for example, with steam, to a temperature equal or higher than the sterilization temperature, which is typically in the range of 115° C.-130° C.

The sterilization process consists of the following five phases:

Sterilization I

During this phase, the preheated process fluid from the storage vessel is introduced into the process vessel. The process fluid exits the bottom of the storage vessel and enters the process vessel through a connection in the suction line of a recirculation pump on the process vessel. This pump extracts the process fluid from the process vessel through a number of outlets at the bottom of the vessel and re-injects the process fluid through a number of inlets at the top of the vessel, typically one inlet per basket. To remove air from the process vessel during filling thereof, a vent valve associated with the process vessel is opened. The duration that the vent valve is open is programmed, typically only on the basis of time and not process fluid level or any other criteria. Steam is injected into the recirculating process fluid to compensate for the temperature loss therein.

Sterilization II

This phase starts from the closing of the vent valve and extends until the desired processing temperature is reached within the process vessel. The connection between the storage vessel and the process vessel remains open until the end of phase Cool I, discussed below.

During the sterilization phases, an overpressure is maintained within the process vessel. This overpressure is provided so that an acceptable pressure differentiation exists between the inside of the containers and the pressure within the process vessel. In some cases the internal pressure of a container is sufficient to cause the container to burst open if an opposing pressure is not provided in the process vessel, but too much opposing pressure may crush or otherwise damage the container. As will be understood, the desired amount of overpressure varies depending on the internal temperature within the nutritional product containers. The level of overpressure in the processing vessel is typically controlled by injecting steam within the storage vessel. If the pressure rises above a program value, such pressure is released by a small vent valve.

Sterilization III

This phase begins when the desired processing temperature within the process vessel has been reached. In this phase a majority of, if not the entire, desired lethality is achieved. At the end of the scheduled process time, the first cooling phase begins.

Cool I

In this phase, a cooling fluid valve in the recirculation circuit is opened and cooling fluid is introduced into the process vessel, with the excess fluid from the process vessel being pushed back up into the storage vessel. This phase ends when the storage vessel is again filled, then the connection between the storage vessel and the process vessel is closed.

Cool II

In this phase the cooling process continues by injection of cooling fluid into the process vessel. The pressure in the process vessel is typically controlled by a drain valve for draining the cooling fluid from the vessel. This phase ends when the cooling duration has been completed.

The foregoing existing retort immersion process has certain drawbacks. For example, it is difficult to control the overpressure in the process vessel especially during the cooling phase and the control of the process fluid level in the process vessel depends on various process factors. As noted above, the Cool I phase is accomplished by injecting cooling water directly into the process vessel. The denser, cooler water creates a differential temperature in the process vessel, thereby forcing warmer water back to the storage vessel via the line which interconnects the storage vessel with the process vessel. The time required to fill the process vessel with cool water and the rate of product cooling is directly associated with the temperature and volume/pressure of available cooling water. This is a difficult transition in a typical immersion process because of the lack of controllable inputs, and the direct method of cooling in the process vessel. As such, a variation in plant water pressure can cause significant pressure oscillation during the Cool I phase. Also, during the Cool I phase, overpressure within the process vessel is supposed to be maintained by pressure and vent valves in the storage vessel. The analog input for the pressure differential loop is generally in the process vessel. The combination of a fixed cooling valve in the process vessel, overpressure in the process vessel being controlled by analog valves in the storage vessel, along with varying water pressure and the changing level of water in the storage vessel can create a challenging control loop situation. Pressure variation within the process vessel can be up to 3-5 psig in the initial stages of cooling when the headspace within the pressure vessel collapses with the injection of cooling water.

Further, there is a risk of water hammer occurring in the transition between the first and second cooling phases, especially if the transition occurs too abruptly, or if there is significant temperature differential in the cooling medium between the end of the first cooling phase and the beginning of the second cooling phase.

Also, process fluid may migrate from the process vessel to the storage vessel during the retort process. As stated above, the process vessel and storage vessel are interconnected. In addition, a valve is used between the two vessels to control the flow therebetween. Nonetheless, process water can migrate from the process vessel back to the storage vessel. This occurs when there is a loss or decrease in steam over pressure in the storage vessel, or if the pressure differential in the storage vessel and process vessel becomes marginal at any time during the retort process. The head pressure from the recirculation pump itself can create this marginal pressure differential between the storage vessel and the process vessel in a low pressure retort process.

In addition, temperature migration can occur from the storage vessel to the process vessel. As noted above, overpressure control in prior art full immersion retort systems is accomplished by utilizing the overpressure steam in the storage vessel. The pressure control is typically achieved by two valves in the storage vessel, commonly referred to as a system pressure valve and a system vent valve. These two valves control the pressure in the storage vessel that in turn controls the pressure in the working drum, since there is an open connection (through a connection valve) that links the process vessel and storage vessel together. A residual water barrier in the storage vessel is utilized to block overpressure steam from porting to the process vessel. That the residual water remains in the storage vessel after the process vessel is filled, is critical. If residual water doesn't remain in the storage vessel so that the overpressure steam is allowed to port through the connection valve between the storage vessel and process vessel, temperature control within the process vessel will be adversely affected and it will not be able to be correctly controlled in the process vessel.

Further, cooling of the nutritional product containers typically occurs with a non-sterile cooling medium, i.e., non-sterile water.

The present invention seeks to address the foregoing drawbacks of the current full or partial retort immersion processes.

### SUMMARY OF THE INVENTION

The present invention pertains to a retort system for sterilizing a work product placed within a process vessel to be filled with heated process fluid. The process fluid is introduced to the process vessel from the storage vessel which is in fluid flow communication with the process vessel through a recirculation system. A heating subsystem heats the process fluid which is initially introduced into the storage vessel and then to the process vessel. A cooling subsystem is utilized to cool the work product after sterilization has been completed. A pressure system is in gas flow communication with the process vessel to maintain the pressure within the process vessel at a desired level during the heating and cooling phases of the retort process by supplying pressurized gas to and withdrawing pressurized gas from the process vessel.

In accordance with a further aspect of the present invention, the process fluid recirculation system includes a recirculation circuit interconnecting in fluid flow communication an outlet at the storage vessel to an inlet at the process vessel, and also interconnecting in fluid flow communication one or more outlets at the process vessel with one or more inlets at the storage vessel.

According to a further aspect of the present invention, the upper portion of the process vessel is in gas flow communication with the upper portion of the storage vessel to permit equalization of the pressure in the process vessel and the storage vessel. A valve may be positioned between the storage vessel and the upper portion of the process vessel to control the fluid flow therebetween.

In accordance with a further aspect of the present invention, spray nozzles may be positioned within the process vessel for directing heated process fluid onto the work product during the sterilization process and also for directing cool process fluid onto the work product during the cooling phase.

In accordance with another aspect of the present invention, during the cooling phase, process fluid may be withdrawn from the process vessel and then cooled and then reintroduced into the process vessel thereby utilizing sterilized process fluid during the cooling phase. The process fluid withdrawn from the process vessel may be cooled under controlled conditions by using a heat exchanger prior to reintroduction of the cooled process fluid back into the process vessel.

In accordance with a further aspect of the present invention, the pressurized gas used to maintain a desired pressure level within the process vessel may be composed primarily of air. One or more valves may be utilized for controlling the level of pressure of the air within the process vessel.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of a first embodiment of the present invention; and

FIG. 2 is a schematic diagram of the second embodiment of the present invention.

### DETAILED DESCRIPTION OF THE PREFERRED EMBODIMENT

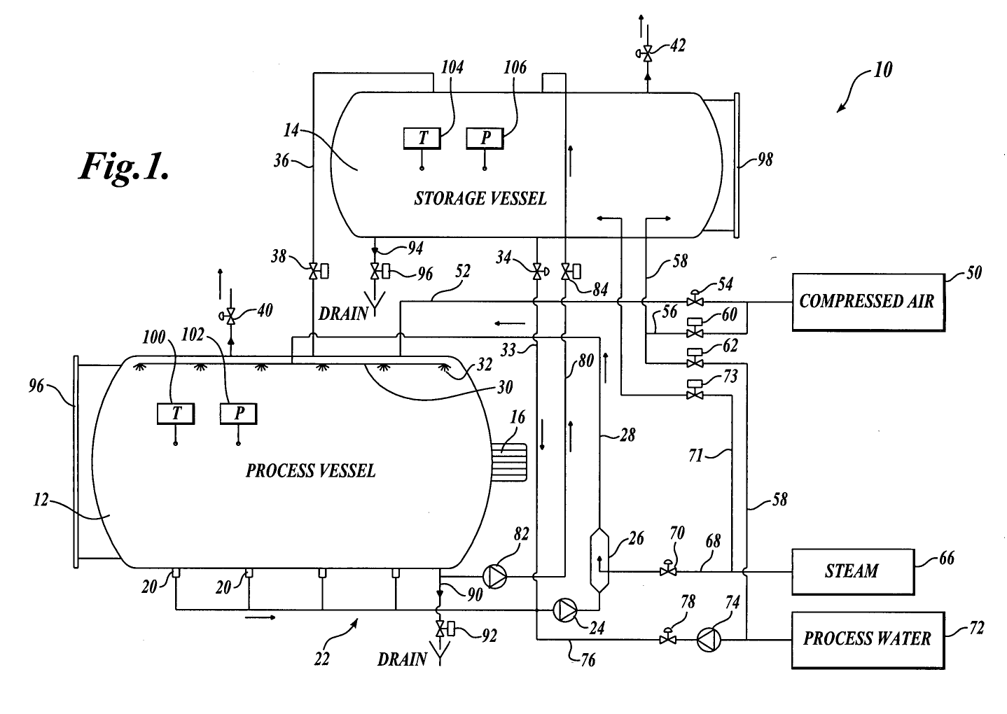

Referring initially to FIG. 1, a retort system 10 is illustrated as including a process vessel 12 in the form of an overpressure retort, and a storage vessel 14 disposed at an elevation above the process vessel. Nutritional products in containers (not shown) are held in baskets or cages (not shown), which in turn are loadable into and unloadable from the process vessel 12. The baskets or containers are rotatable end-over-end by a reel apparatus or drum (not shown), disposed within the process vessel 12 by a variable speed drive motor 16, schematically illustrated in FIG. 1.

The process vessel is filled with preheated working medium or process fluid (typically water) from the storage vessel 14. After the process vessel 12 is filled with process fluid, the fluid is subsequently continuously circulated through the process vessel during the entire retort process. To this end, one or more outlet openings 20 at the bottom of process vessel are connected to a recirculation circuit 22. A pump 24 disposed within the recirculation circuit re-circulates the process fluid from the bottom of the process vessel via a fluid injector 26 (e.g., steam injector) which may be a low-noise injector, through a line 28 back into vessel 12 via one or more spray lines or pipes 30 extending along the length of the process vessel. It will be appreciated that a network of such pipes or lines 30 may be utilized. Fluid outlets or nozzles 32 are disposed along the length of the pipes/lines 30 to direct the working medium (process fluid) toward the containers disposed within the process vessel.

The bottom of the storage vessel 14 is connected to the inlet of the recirculation pump 24 by line or conduit 33 and control valve 34. The headspaces (upper portions) of the storage vessel 14 and process vessel 12 are interconnected through an equalizer conduit or line 36 and an associated valve 38.

Overpressure can be applied to the process vessel 12 during the complete processing cycle. The overpressure can be programmed in accordance with the process requirements and may be controlled by an automatic proportional valve 40 in flow communication with the upper portion of process vessel 12 to release the pressure within the head space of process vessel 12 and by a pressurized gas (compressed air) source 50 connected to process vessel 12 via line 52 and associated valve 54, which may be an automatic proportional valve, to increase the pressure. When the valve 38 in the connection line 36 is closed, the pressure in the storage vessel is controlled by the valve 42 positioned at the upper portion of storage vessel 14 to release the pressure therein and by the pressurized gas (compressed air) source 50 to the storage vessel via line 56 and associated control valve 60 which intersects line 58, which directs process fluid to the storage vessel.

The process fluid may be heated by direct injection of heated or super-heated heating medium (e.g., steam) into the recirculation circuit 22. The heating medium from a source 66 is routed to an injector 26 via line 68. A control valve 70, which may be an automatic proportional valve, is interposed in line 68 for controlling the flow of heating medium between source 66 and injector 26. As discussed above, the injector is disposed within recirculation line 28 of the process vessel. Heating medium (e.g., steam), for initially heating the process fluid, from source 66 is routed to storage vessel 14 via line 71, which is interconnected between line 68 and vessel 14. A control valve 73 is interposed within line 71.

As noted above, the storage vessel 14 is connected to a process fluid supply 72 through line 58 and valve 62. Correspondingly, the process fluid supply 72 is connected to the process vessel through a booster pump 74 disposed within line 76 of the recirculation circuit leading to line 28. A control valve 78 is disposed downstream of the booster pump 74 within line 76.

The bottom of the process vessel 12 is connected to the top portion of the storage vessel 14 via a line or conduit 80 through the use of a pump 82 disposed within the line. A control valve 84 is also disposed within line 80 downstream of pump 82.

Process fluid can be drained from the process vessel 12 via drain line 90 and drain valve 92. Correspondingly, process fluid can be drained from storage vessel 14 via drain line 94 and associated valve 96. A level of process fluid in process vessel 12 may be measured by a level probe 96 connected to the process vessel shown in FIG. 1. Correspondingly, the level of process fluid within storage vessel 14 may be measured by a level probe 98 connected to the storage vessel shown in FIG. 1. The process vessel 12 is also equipped with a temperature measuring device 100 and a pressure measuring device 102. Correspondingly, the storage vessel is equipped with a temperature measuring device 104 and a pressure measuring device 106. Such temperature and pressure measuring devices are standard articles of commerce and will not be described with particularity herein.

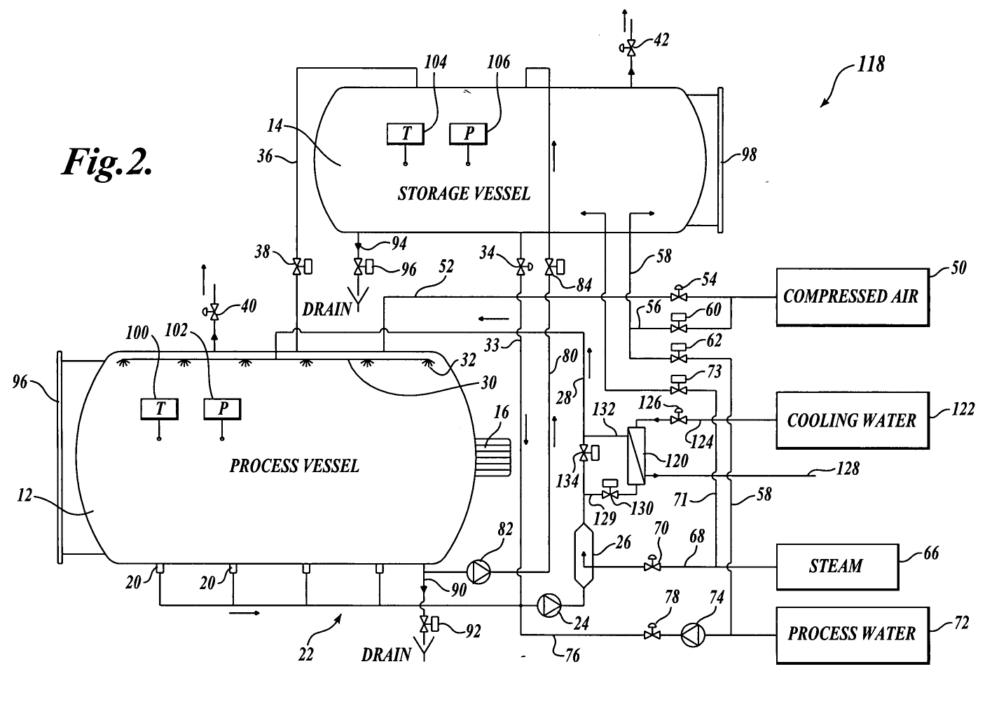

FIG. 2 illustrates an alternative embodiment to the present invention which is similar to FIG. 1 but utilizes an indirect cooling system in combination with spray cooling in the process vessel. The components of the present invention shown in FIG. 2 that are the same or very similar to that in FIG. 1 are given the same part numbers.

In FIG. 2 the retort system 118 includes a heat exchanger 120 that receives a cooling medium (for example, water) from a cooling medium source 122 via inlet line 124, and associated valve 126. The cooling medium is removed from the heat exchanger 120 via outflow line 128. The heat exchanger 120 is interconnected to recirculation line 28 via inlet line 129, wherein is disposed a control valve 130, and via outlet line 132. A diverter valve 134 is disposed in line 28 between the locations that the lines 129 and 132 intersect line 28 for use in directing some or all of the recirculating process fluid through the heat exchanger 120.

The working medium (process fluid) from the pressure vessel 12 is cooled by being diverted by valves 130 and 134 in recirculation circuit 22 through inlet line 128 to the heat exchanger 120 and then routed back to the process vessel through the line 132. Such cooled process fluid may be introduced into the process vessel through the pipes 30 and nozzles/outlet 32. It will be appreciated that in FIG. 2 the cooling medium is sterilized since it is composed of re-circulated process fluid from the process vessel 12.

Referring back to FIG. 1, the retort immersion process of the present invention consists of several phases, including the following:

Fill and Preheat Storage Vessel

In this phase, the fluid fill valve 62 in line 58 is opened to allow the process fluid from source 72 to enter the storage vessel. The level of the process fluid is monitored by use of the level probe 98. Once a desired minimum level of process fluid is reached within the storage vessel 14, valve 73 is opened to allow heating medium (e.g., steam) from source 66 to also flow into the storage vessel, thereby to start heating the process fluid in the storage vessel introduced via line 58. The heating medium is injected into the storage vessel through low noise injectors (not shown) disposed in the bottom of vessel 14. Such injectors are articles of commerce, and thus will not be described with particularity herein.

As the storage vessel 14 is filled with process fluid, the pressure therein increases, and once a preprogrammed pressure level is reached, as measured by pressure measuring device 106, the pressure relief valve 42 is opened and controlled to maintain a target pressure level within the storage vessel. The fill valve 62 is closed once the desired fill level of process fluid is reached within the vessel 14. Also, once the temperature of the process fluid within storage vessel 14 has been reached, as measured by temperature measuring device 104, the valve 73 is also closed.

Process Vessel Fill (Sterilization I)

Once the process vessel 12 is loaded with baskets or cages, that contain nutritional product containers, the door (not shown) to the vessel is closed. Thereafter, the process vessel is filled with process fluid from the storage vessel 14. To this end, fill valve 34 in line 32 is slowly opened to route the superheated process fluid from the storage vessel 14 to the bottom of the process vessel 12 via line 33 of the recirculation circuit 22 which is in fluid flow communication with inlets/outlets 20 at the bottom of the process vessel. As the superheated process fluid enters the process vessel (which is at atmospheric pressure), the process fluid flashes and the pressure within the vessel begins to increase.

The valve 38 in equalizer line 36 is then opened so that the pressure in the storage vessel 14 and in the process vessel 12 can equalize. In this regard, air or other gas displaced from the process vessel 12 is routed to the storage vessel as the process vessel fills and the level of the process fluid in the storage vessel drops. Valve 38 remains open during the remainder of the sterilization process, discussed below (Come-Up, Cook and Cool). As such, there is no pressure differential between the process vessel and storage vessel.

When a minimum process fluid level is reached in the process vessel (this level still being below the bottom of the baskets), the recirculation pump 24 is started so that the process fluid is also injected and distributed over the top of the baskets via outlets/nozzles 32 disposed along the lines 30. This facilitates the uniform heating of the containers within the process vessel during the fill phase. The recirculation pump 24 operates continuously during the entire sterilization cycle, until the end of the cooling cycle.

When the recirculation flow is initiated, the valve 70 is opened to inject heating medium into the recirculating process fluid in line 28 to compensate for the temperature loss in the process fluid as it comes into contact with the containers and the process vessel.

The rotation of the reel or drum within the process vessel is started according to a preprogrammed speed. The fill valve 34 is closed when the target level of the process fluid in the process vessel 12 is reached. This is the end of the fill phase. At this point, a certain amount of process fluid will remain in the bottom of the storage vessel 14.

As noted above, overpressure in the process vessel is achieved by use of pressurized gas, typically air. The level of the overpressure is controlled by controlling the pressurized gas valve 54 as well as the pressure relief valve 40 of the process vessel. It will be appreciated that the large volume of pressurized gas in the storage vessel acts as a buffer and allows for very accurate gas pressure control within the process vessel.

Rather than using only the storage vessel to store pressurized gas used for controlling the overpressure within the process vessel, such pressurized gas may be stored in a separate accumulator that is independent from the process vessel. The accumulator may be positioned between the storage vessel and the process vessel, and connected in gas flow communication with both the process vessel and the storage vessel. The accumulator may utilize a pressure relief valve similar to valve 42, as well as valves positioned in a line connecting the process vessel with the accumulator, similar to valve 38, as well as a valve positioned in a line connecting the accumulator with the storage vessel.

Come-Up (Sterilization II)

During the temperature come-up phase, the temperature and pressure within the process vessel 12 are independently controlled by proportional valve 70 for the heating medium, as well as valve 54 for pressurized gas and proportional valve 40 to relieve the pressure build-up within the process vessel. The come-up phase can be divided into different steps to achieve the temperature and air overpressure profiles that is optimized for the particular containers and for the particular nutritional products within the containers. By controlling the proportional valve 70, heating medium is injected into the recirculation circuit 22 via a injector 26, thereby providing a pre-programmed temperature profile. Also, by controlling the proportional valves 54 for pressurized gas and 40 for pressure relief, the overpressure within the process vessel may also be controlled to achieve the pre-programmed pressure profile.

The direct injection of heating medium (steam) from source 66 into the circulation circuit 22 results in condensate that increases the level of process fluid in the process vessel 12. The level of the process fluid in the process vessel can be controlled by use of the level probe 96. The excess process fluid is transferred to the storage vessel 14 with the use of pump 82 and also by controlling the operation of valve 84 within line 80.

Cook Phase (Sterilization III)

The temperature, pressure, and process fluid level within the process vessel 12 are controlled during the cook phase in the same way as during the come-up phase discussed above, according to the pre-programmed steps for the cook phase. Applicant has found that the direct injection of heating medium via injector 26, combined with the recirculation of process fluid through recirculation circuit 22, and the rotation of the drum within the process vessel, very good temperature distribution occurs within the process vessel.

Cooling Phase

The cooling of the containers within the process vessel may be achieved by direct injection of non-heated process medium from source 72 into the recirculation circuit 22 through the use of booster pump 74 and associated control valve 78. The cooling medium is delivered through line 76 into the recirculation pump 24 of the recirculation circuit 22, where the cooling medium mixes with the process fluid drawn from the bottom of the processing vessel 12 by pump 24.

The cooling phase may consist of two subphases, designated as Cool-I and Cool-II.

Cool-I

At the end of the cook phase (sterilization III), the heating medium valve 70 is closed and the pump 74 is started and the valve 78 is opened to draw non-heated process fluid from source 72. During this subphase, the gas overpressure within process vessel 12 is still controlled with the proportional valve 54 in pressurized gas line 52 and the pressure relief valve 40. In this manner, the transition from the cook phase to the cooling phase occurs in a controlled manner, thereby avoiding sudden temperature or pressure drops that may damage sensitive containers within the process vessel.

During the Cool-I phase, the process fluid level within the process vessel is controlled by controlling pump 82 and associated valve 84 that returns the excess process fluid from the process vessel to the storage vessel. This helps avoid any pressure variation within the process vessel since the head spaces within the process vessel and the storage vessel are connected to each other, as described above. The rate of cooling of the contents of the process vessel is controlled according to pre-programmed cooling steps by controlling the opening of the proportional cool process medium valve 78.

At the end of the cook phase and at the beginning of the cooling phase, the circulation pump 24 can also be utilized to return the process fluid from the process vessel 12 to the storage vessel 14, thereby reducing the time required to remove the process fluid from the process vessel. Of course, appropriate piping and valves from the recirculation line 28 to the storage vessel 14 would be required. Also during use of the recirculation pump for this purpose, the pressure and temperature within the process vessel would be monitored to make sure that the desired pressure and temperature profiles are achieved and maintained.

Cool-II

This cooling subphase begins when a desired initial, pre-filled fluid level in the storage vessel is reached. As described above, this can be determined by use of the process fluid level probe 98. When the desired process fluid level in the storage vessel is reached, valve 84 is closed and the pump 82 is switched off. The level of process fluid within the process vessel is now controlled by opening and closing the drain valve 92, which is disposed within line 90 leading from the lower portion of the process vessel. During the Cool-II subphase, the process fluid inlet valve 78 is controlled according to a chosen sequence of cooling steps designed to achieve the desired results. The gas overpressure within the process vessel is still controlled with the portional valve 54 in pressurized gas in line 52, and by the pressure relief valve 40, according to the pre-programmed cooling steps.

End of Cycle

Upon completion of the cooling phase, the cool process medium inlet valve 78 is closed, and the booster pump 74 is switched off. Also, the process fluid re-circulation pump 24 is switched off while the drum, with the baskets therein, is rotated to the basket drain position. Also at the end of the cycle, the valve 38 in the interconnection line 36 between the process vessel and the storage vessel is closed.

The process fluid is drained from the vessel 12 by opening the drain valve 92 in line 90. To hasten or facilitate draining of the process vessel, the gas overpressure at the end of the cooling phase can be maintained by continuing to inject pressurized gas into the process vessel by a valve 54 and line 52.

Once the process vessel has been drained to a target level, the residual pressure within the process vessel 12 is released by opening valve 40. When the pressure within the process vessel is back down to atmosphere pressure, the drum within the processing vessel is rotated to its horizontal “home” position. Thereupon, an “end of cycle” signal informs the retort operator that the cycle has been completed and that the door to the process vessel can be opened to remove the containers therein.

Reheating of the Process Fluid

The reheating of the process fluid within the storage vessel for the next cycle commences directly at the end of the cooling cycle once the valve 38 in equalizing line 36 has been closed. Initially the pressure within the storage vessel 14 is increased to the target value by opening the pressurized gas valve 60 within line 56, thereby to route pressurized gas from source 50 through lines 56 and 58 into the storage vessel. Thereafter, the valve 73 and line 71 is opened to reheat the process fluid within the storage vessel until the target temperature is reached.

Indirect Cooling Process

As discussed above, FIG. 2 illustrates the present invention, wherein indirect cooling of the process vessel is utilized. In this regard, with respect to the retort system 118 shown in FIG. 2, the Fill and Pre-Heat phase, the Process Vessel Fill phase, the Come-Up phase, and the Cook phase may be the same as described above with respect to system 10. The indirect cooling system provides a cooling process for the process vessel and employs sterile cooling fluid. Two phases are used, an Initial Cool Phase and a Spray Cool Phase.

Initial Cool Phase

At the end of the Sterilization phase, the heating medium valve 70 is closed and the valve 130 in line 129 is opened, while the diverter valve 134 is operated so that part of the recirculation process fluid flow is passed through the primary side of heat exchanger 120. The cooling medium valve 126 is then opened to pass cooling medium in counterflow to the secondary side of the heat exchanger 120. This controlled and gentle transition from cooking to cooling avoids sudden temperature or pressure drops in the process vessel that may damage sensitive food containers therein.

During the Initial Cool Phase, the gas overpressure within the process vessel is controlled with the proportional valve 54 in pressurized gas line 52 and by relief pressure valve 40 of the process vessel, according to a pre-programmed profile. The rate of cooling of the process fluid within the process vessel is controlled according to the pre-programmed cooling steps by controlling the cooling medium flow through the heat exchanger 120 through use of the proportional valve 126.

Spray Cool

During this phase, when the temperature of the process fluid within the process vessel 12 has dropped to a certain predetermined value (for instance, 220 degrees Fahrenheit), most of the process fluid has been transferred back to the storage vessel by pump 82. As the process fluid is pumped back to the storage vessel, the gas in the head space of the storage vessel 14 is returned to the process vessel 12 via equalizer line 36, since at that point valve 38 is still open. When the target process fluid level within the storage vessel 12 is reached, the pump 82 is switched off and the valves 38 and 84 are closed. At this point, the reheating of the process fluid in the storage vessel can commence, as described above.

The remaining process fluid is drained from the process vessel by operation of outlet valve 92 until the minimum level needed for spray cooling is reached. At this point, only a small amount of the process fluid remains at the bottom of the process vessel. The process fluid is continuously re-circulated and re-injected through the spray nozzles 32, whereby the process fluid is cooled down through the heat exchanger 120. In this way, warm process fluid is returned to the storage vessel 14 while the thermal mass during the remainder of the spray-cool phase is reduced to an absolute minimum.

The gas overpressure within the process vessel 12 is controlled with the proportional valve 54 in the pressurized gas line 52 and the relief valve 40, according to a pre-programmed profile. Due to the large gas volume in the process vessel, the overpressure control can be very accurate.

End of Cycle

The End of Cycle phase for the indirect cooling system 118, shown in FIG. 2, is the same as described above with respect to system 10. However, since the water level in the process vessel is already very low, the time required to drain the remainder of the process fluid from the process vessel is much shorter.

It will be appreciated that, through the present invention, significant drawbacks of existing immersion retort processes are overcome. For example, as explained above, in existing overpressure immersion systems, it is difficult to control the pressure within the process vessels, especially during the cooling phases of the retort process. However, through the present invention, cooling is accomplished by utilizing a separate pump to control the return of the process fluid from the process vessel to the storage vessel. As a result, the time that cooling fluid is introduced into the process vessel, as well as the pressure and volume of the cooling fluid, may be more precisely controlled. In addition, the overpressure within the process vessel is more accurately controlled by use of compressed gas (air) in both the process vessel and the storage vessel. Also, in the indirect cooling method described above, by using a heat exchanger, the temperature of the cooling fluid can be accurately controlled.

In addition, by moving substantially all of the process fluid from the process vessel during cooling, cooling of the nutrient products within the process vessel can be accomplished by water spray, which is more efficient then having to immerse the nutrient product containers in a large volume of cooling water.

Also, the present invention advantageously utilizes air, not steam, for overpressure control. The pressurized air is not a heat source and will not affect the temperature control in the process vessel. This reduces the possibility of temperature migration of overpressure steam from the storage vessel to the process vessel, described above.

In addition, the present invention addresses the water migration problem, described above with respect to current retort immersion systems. Because the process vessel and storage vessel are truly isolated from each other, water migration cannot occur during the cook phase. As a result, it is possible to accurately control the pressure within the process vessel of the present invention, even at minimal overpressures.

While the preferred embodiment of the invention has been illustrated and described, it will be appreciated that various changes can be made therein without departing from the spirit and scope of the invention.

1. A retort system for sterilizing a work product, comprising:
(a) a process vessel for receiving work product and for receiving process fluid to sterilize the work product within the process vessel;
(b) a storage vessel for storing the process fluid;
(c) a process fluid recirculation system, comprising a recirculation circuit interconnecting in fluid flow communication an outlet at the storage vessel to an inlet at the process vessel and also interconnecting an outlet at the process vessel with an inlet at the storage vessel;
(d) a heating subsystem for heating the process fluid;
(e) a cooling subsystem for supplying cooling process fluid to the process vessel; and
(f) a pressure system in gas flow communication with the process vessel to maintain the pressure within the process vessel at desired levels during the retort process.

2. The retort system according to claim 1, wherein gas is supplied to and withdrawn from the process vessel during the retort process.

3. The retort system according to claim 2, wherein an upper portion of the process vessel is connected to an upper portion of the storage vessel to permit equalization of the pressure in the process vessel and the storage vessel.

4. The retort system according to claim 3, further said pressure system comprising a valve positioned between the upper portion of the storage vessel and the upper portion of a process vessel to control fluid flow therebetween.

5. The heating subsystem according to claim 1, comprising a source of heating medium for heating the process fluid supplied to the recirculation system.

6. The retort system according to claim 5, further comprising spray nozzles positioned within the process vessel for directing the heated process fluid onto the work product disposed within the process vessel.

7. The retort system according to claim 1, further comprising spray nozzles, positioned within the process vessel for directing the heated process fluid and cooled process fluid onto the work products disposed within the process vessel.

8. The retort system according to claim 1, wherein the cooling subsystem comprises a supply of cooling process fluid and an inlet for introducing the cooling process fluid into the recirculation system.

9. The retort system according to claim 8:
further comprising spray outlets positioned within the process vessel; and
wherein the recirculation system directing the cooling process fluid to the spray outlets.

10. The retort system according to claim 8:
wherein the source of the cooled process fluid is the process fluid within the pressure vessel; and
wherein the cooling subsystem further comprising a heat exchanger for cooling the process fluid from the process vessel prior to reintroduction into the process vessel.

11. The retort system according to claim 10, wherein the cooling process fluid is sourced from the process vessel and then once extracted from the process vessel, the cooling process fluid is cooled to a desired temperature.

12. The retort system according to claim 11, wherein the process fluid derived from the process vessel is cooled in a heat exchanger.

13. The retort system according to claim 8, wherein the source of cooling process fluid is process fluid extracted from the process vessel and then subsequently cooled to the desired temperature.

14. The retort system according to claim 13, wherein cooling subsystem further comprising a heat exchanger for cooling the process fluid from the process vessel prior to reintroduction of the cooled process fluid into the process vessel.

15. The retort system according to claim 1, wherein the pressure system maintains the pressure in the process vessel at a level above atmosphere pressure during at least a portion of the sterilization process of the work product within the process vessel.

16. The retort system according to claim 15, wherein the pressure system is also in gas flow communication with a volume of pressurized gas that functions as a buffer for a pressurized gas within the process vessel.

17. The retort system according to claim 15, wherein the gas utilized in the pressure system to provide overpressure to the process vessel comprises air.

18. The retort system according to claim 17, wherein the pressure system further comprising one or more valves for controlling the level of overpressure within the process vessel.

19. The retort system according to claim 17, wherein the pressure system is in airflow communication with a volume of pressurized air that is separate and distal from the pressurized air within the process vessel, wherein said separate volume of pressurized air acts as a buffer for the pressurized air within the process vessel.

20. The retort system according to claim 19, wherein the separate volume of pressurized air is located within the storage vessel.

21. The retort system according to claim 17, wherein the air utilized in the pressure subsystem is in air flow communication with the upper portion of the storage vessel during at least part of the sterilization process.

22. The retort system according to claim 15, wherein the gas utilized to achieve overpressure within the process vessel is in gas flow communication with an upper portion of the storage vessel during at least part of the sterilization process performed in the process vessel.

23. The retort system according to claim 15, wherein the pressure system comprising one or more valves for controlling the level of overpressure within the process vessel created by the pressurized gas.

24. The retort system according to claim 1, wherein air is utilized by the pressure system as the gas for maintaining the pressure within the process vessel at desired levels during the retort process.

25. The retort system according to claim 24, wherein the air utilized to maintain the overpressure in the process vessel is in air flow communication with the storage vessel during at least part of the sterilization process.

26. An overpressure retort system comprising:
(a) a process vessel for receiving containers of nutritional products therein and for also receiving process fluid for sterilizing the nutritional product within the containers when within the process vessel;
(b) a process fluid storage vessel for receiving and storing process fluid therein;
(c) a process fluid recirculation system, comprising a recirculation circuit interconnecting the storage vessel and the pressure vessel for directing process fluid to and from the storage vessel, to and from the process vessel and between the storage and process vessels;
(d) a heating system for heating the process fluid and introducing the heated process fluid into the storage vessel and/or process vessel;
(e) a cooling system for supplying process fluid to the process vessel for cooling the sterilized nutritional products therein; and
(f) an overpressure system for supplying pressurized gas to the process vessel and the storage vessel, to maintain the pressure in the process vessel at the desired levels during the sterilization process.

27. The overpressure retort system according to claim 26, wherein said overpressure system further comprising connecting the headspace area within the process vessel is in gas flow communication with the headspace area within the storage vessel whereby the storage vessel functions as an accumulator vessel for the gas utilized to achieve the desired overpressure within the process vessel.

28. The overpressure retort system according to claim 27, wherein said overpressure system further comprising a valve positioned between the headspace area within the process vessel and the headspace area of the storage vessel to control the gas flow therebetween.

29. The overpressure retort system according to claim 26, further comprising spray nozzles positioned within the process vessel for directing the heated process fluid onto the nutritional products disposed within the process vessel.

30. The overpressure retort system according to claim 29, wherein said spray nozzles also would direct process fluid for cooling the sterilized nutritional products onto the containers of the sterilized nutritional products.

31. The overpressure retort system according to claim 30, wherein the source of process fluid for cooling the sterilized nutritional products is process fluid extracted from the process vessel and then subsequently cooled to a desired temperature.

32. The overpressure retort system according to claim 31, wherein the process fluid derived from the process vessel is cooled in a heat exchanger.

33. The overpressure retort system according to claim 26:
wherein the source of process fluid for cooling the sterilized nutritional products is the process fluid within the process vessel; and
wherein the cooling system further comprising a heat exchanger for cooling the process fluid from the process vessel prior to reintroduction into the process vessel.

34. The retort system according to claim 26, wherein the overpressure system maintains the pressure in the process vessel at a level above atmospheric pressure during at least a portion of the sterilization process of the nutritional products within the process vessel.

35. The retort system according to claim 34, wherein the overpressure system further comprises a volume of pressurized gas in gas flow communication with the process vessel to act as a buffer for the pressurized gas within the process vessel.

36. The overpressure retort system according to claim 35, wherein the gas utilized to achieve overpressure within the process vessel is in gas flow communication with an upper portion of a storage vessel during at least a portion of the sterilization process performed in the process vessel.

37. The overpressure retort system according to claim 26, wherein the gas utilized in the overpressure system to provide overpressure to the process vessel comprises air.

38. The overpressure retort system according to claim 37, further comprising a volume of pressurized air located separately and distally from the process vessel, said volume of pressurized air in airflow communication with the process vessel, said volume of pressurized air serving as the buffer for the pressurized air within the process vessel.

39. The overpressure retort system according to claim 26, wherein the overpressure system comprising one or more valves according to the level of overpressure within the process vessel created by the pressurized gas.

40. The overpressure retort system according to claim 39, wherein said overpressure system further comprises an accumulator in gas flow communication with the process vessel to store pressurized gas used to maintain the pressure in the process vessel at desired levels during the sterilization process.

41. The overpressure retort system according to claim 40, wherein said accumulator comprises a portion of the process fluid storage vessel.

42. A method for sterilizing nutrient products disposed in containers utilizing an overpressure retort system having a process vessel and a storage vessel, wherein the process vessel and storage vessel are interconnected in fluid flow communication therebetween, the process comprising:
(a) placing containers of the nutrient products within the process vessel;
(b) filling the storage vessel to a desired level with heated process fluid;
(c) introducing the heated process fluid from the storage vessel to the process vessel, leaving a desired headspace within the process vessel;
(d) recirculating the process fluid within the process vessel by drawing the process fluid from the process vessel, reheating the withdrawn process fluid as needed, and reintroducing the heated process fluid back into the process vessel;
(e) supplying gas at a desired pressure to the headspace of the process vessel to maintain a desired overpressure within the process vessel;
(f) after completion of the sterilization process, withdrawing the process fluid from the process vessel and routing such withdrawn process fluid back to the storage vessel;
(g) cooling the nutrient product containers within the process vessel with process fluid; and
(h) maintaining a desired overpressure within the process vessel during the sterilization process by controlling the pressure of the compressed gas supplied to the headspace of the process vessel.

43. The method according to claim 42, wherein the headspace of the process vessel is in gas flow communication with a volume of pressurized gas that is separate and distal from the volume of pressurized air in the process vessel, wherein such separate volume of pressurized gas acts as a buffer for the headspace of the process vessel.

44. The method according to claim 43, wherein the separate volume of pressurized air is located in the storage vessel.

45. The method according to claim 42, wherein the heated process fluid is sprayed onto the containers of nutrient products in the process vessel.

46. The method according to claim 42, wherein the process fluid during cooling of the nutrient products within the process vessel is sprayed onto the containers of the nutrient products.

47. The method according to claim 42, wherein the cooling of the nutrient products is achieved by withdrawing process fluid from the process vessel, cooling the withdrawn process fluid and then reintroducing the cooled process fluid back into the process vessel;

48. The method according to claim 47, wherein the process fluid withdrawn from the process vessel is cooled within a heat exchanger.
